(19) United States
(12) Patent Application Publication
(10) Pub. No.: US 2025/0018086 A1
(43) Pub. Date: Jan. 16, 2025

(54) ALIGNED AND MULTICHANNEL PERIPHERAL NERVE CONDUIT AND PREPARATION METHOD THEREOF

(71) Applicant: THE HONG KONG UNIVERSITY OF SCIENCE AND TECHNOLOGY, HONG KONG (CN)

(72) Inventors: ZHENGTANG LUO, HONG KONG (CN); YUTING CAI, HONG KONG (CN)

(21) Appl. No.: 18/349,175

(22) Filed: Jul. 10, 2023

(51) Int. Cl. A61L 27/08; B82Y 5/00; C01B 32/194; C01B 32/198

(52) U.S. Cl. CPC A61L 27/08; B82Y 5/00; C01B 32/194; C01B 32/198

(57) ABSTRACT

An aligned and multichannel peripheral nerve conduit, including Polyvinyl alcohol (PVA) and graphene oxide (GO) with aligned and multichannel design for peripheral nerve repair. 3D printing technology with assembly is utilized to make a specific mold to get a multichannel design to mimic natural peripheral nerves. Directional freeze casting is used to form an aligned PVA/GO hydrogel structure to guide cell growth.

### TECHNICAL FIELD

The invention relates to the technical field of peripheral nerve repair, in particular to aligned and multichannel peripheral nerve conduit and preparation method thereof.

### BACKGROUND

Peripheral nerve injury (PNI), which is mainly caused by accident or sports, can lead to impaired sensory and motor function in patients. Due to the complexity of nerve tissue structure and function, PNI is still a common but intractable disease. In the 21st century, more than 5 million new cases have been reported in the whole world. In severe cases, patients will permanently lose their moving ability when the nerve trunk is completely broken. When the gap of the damaged nerve is larger than 10 mm, It is impossible to achieve complete reinnervation if only by relying on its self-healing potential. In clinical treatment, the most effective way is autologous nerve transplantation. However, due to the limited source of donor's nerves and the mismatch of donor and recipient nerves, its subsequent development has been restricted. Since the 1940s, when artificial catheters have first been used to bridge disconnected neural structures, the development of polymer materials and tissue engineering provided new ideas for peripheral nerve conduits. Biomaterials such as collagen, silk fibroin, and chitosan are widely applied to promote functional recovery of injured nerves owing to good biocompatibility. The US Food and Drug Administration (FDA) has approved some of them for clinical application, and some products in the market show positive results in clinical applications. However, all of them are single tubular structures which are unacceptable in repairing large gaps of nerve defects. The single tubular structure cannot imitate the natural nerves that the multi-hollow lumen can guide nerve growth and innervation, which further results in the failure of nerve function recovery. In addition, recent research work illustrates that scaffolds made of aligned nanofibers are promising for nerve regeneration and that particular topographical cues can guide and boost axon elongation. Some studies have proved that aligned nanofibers can stimulate and promote Schwann cell orientation and axon growth in vitro. However, electrospinning technology as the key step for aligned fibers fabrication is complicated and time-consuming. Additionally, the selection of materials is limited because most hydrogel is not applicable. New technology to fabricate conduits with aligned structures is imperative.

### SUMMARY

In one aspect, a method of preparing aligned and multichannel peripheral nerve guidance conduit is provided, where the method includes directional freeze casting and specifical mold. The details are shown below:
S1: Graphene oxide (GO) was prepared by a modified Hummers method, which chemically separates the graphite interlayer by adding functional groups and then dispersing the GO into 100 ml of the deionized (DI) water to obtain GO solution (0.5 mg/mL);
S2: 10 g Polyvinyl alcohol (PVA) was dissolved into 100 mL GO solution (˜80° C.) with strong stirring to obtain a homogeneous PVA/GO solution;
S3: Pouring the prepared mixed solution into the mold and set the whole mold above liquid nitrogen so that the bottom copper sheet was just enough to touch the liquid nitrogen. The ice nucleus would grow vertically and generate ice pillars in parallel. Later, the sample was thawed at room temperature. The freeze-thawing cycles were repeated another four times. Finally, the frozen samples were freeze-dried.
S4: The obtained dry conduits were then annealed at 100° C. for 30 minutes, followed by soaking into deionized water until equilibrium and mold removal, resulting in the conduit with aligned structures.

In another aspect, a specific mold is fabricated by 3D printing technology, and the design parameter is given as follows.
S1: Acrylonitrile butadiene styrene (ABS) models were fabricated by a 3D printing method, including a pipe (inner diameter: 3 mm, outer diameter: 5 mm, height: 50 mm), 4 cylinders (diameter: 0.5 mm, height: 70 mm) and a disc (diameter: 5 mm, thickness: 1 mm).
S2: Assembly of printed parts. 1. A pipe was glued vertically to the copper sheet. 2. Four cylinders were penetrated vertically into a disc. The four penetration points formed a square (Side length: 2 mm); the center of the square and the center of the circle coincided. 3. Four cylinders with a disc are put into the prepared pipe, and the disc can cover the whole pipe.

In a further aspect, the morphology of conduits is observed by scanning electron microscope (SEM), and mechanical properties were also measured.

### BRIEF DESCRIPTION OF THE DRAWINGS

FIG. 1A shows a schematic diagram of a model printed using 3D printing.

FIG. 1B shows the assembled mold.

FIG. 2A represents the PVA/GO solution in mold.

FIG. 2B shows the alignment direction of PVA chains and GO sheets after directional freezing.

FIG. 2C specifically demonstrates how directional freezing induces the alignment of PVA and GO by generating ice crystals.

FIG. 3A illustrates the SEM image of the cross-section of aligned and multichannel PVA/GO conduit.

FIG. 3B illustrates the SEM image of magnified cross-section of aligned and multichannel PVA/GO conduit.

FIG. 3C illustrates the SEM image of magnified longitudinal section of the aligned and multichannel PVA/GO conduit.

FIG. 4A is the tensile stress-strain curves of anisotropic PVA/GO aerogel with different tensile directions and isotropic PVA/GO aerogel.

FIG. 4B is the Young's modulus of anisotropic PVA/GO aerogel with different tensile directions and isotropic PVA/GO aerogel.

### DESCRIPTION OF EMBODIMENTS

Various embodiments are described below. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not apparent to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Herein, we report a novel design and fabrication of an aligned and multichannel peripheral nerve conduit with a specific mold. The synthesis process includes directional freeze casting and freeze drying, below are the details for the synthesis process:
S1: Graphene oxide (GO) was prepared by a modified Hummers method, which chemically separates the graphite interlayer by adding functional groups and then dispersing the GO into 100 ml of the deionized (DI) water to obtain GO solution (0.5 mg/mL);
S2: 10 g Polyvinyl alcohol (PVA) was dissolved into 100 mL GO solution (˜80° C.) with strong stirring to obtain a homogeneous PVA/GO solution;
S3: Pouring the prepared mixed solution into the mold and setting the whole mold above liquid nitrogen. That bottom copper sheet was just enough to touch the liquid nitrogen. The ice nucleus would grow vertically and generate ice pillars in parallel. Later, sample was thawed at room temperature. The freeze-thawing cycles were repeated another four times. Finally, the frozen samples were freeze-dried.
S4: The obtained dry conduits were then annealed at 100° C. for 30 minutes, followed by soaking into deionized water until equilibrium and mold removal, resulting in the conduit with aligned structures.

The multichannel structure design of conduits can mimic the inner structure of autologous nerves, enhance axon extension and guide directional reinnervation. The multichannel strategy of this conduit is achieved by specific mold through 3D printing. The fabrication and parameters of the mold are listed as follows.
S1: Acrylonitrile butadiene styrene (ABS) models were fabricated by 3D printing method including a pipe (inner diameter: 3 mm, outer diameter: 5 mm, height: 50 mm), 4 cylinders (diameter: 0.5 mm, height: 70 mm) and a disc (diameter: 5 mm, thickness: 1 mm).
S2: Assembly of printed parts. 1. A pipe was glued vertically to the copper sheet. 2. Four cylinders were penetrated vertically to a disc. The four penetration points formed a square (Side length: 0.5 mm); the center of the square and the center of the circle coincided. 3. Four cylinders with a disc are put into prepared pipe, and the disc can cover the whole pipe.

The aligned structure with multichannel design can facilitate synergistic effects in directed axonal extension and Schwann cell migration. The new guided hydrogel nerve conduits combined with two guided strategies may provide a new avenue to enhance angiogenic and regenerative capacity for nerve repair.

### EMBODIMENTS

### Embodiment 1. Design and Fabrication of Mold by 3D Printing Technology

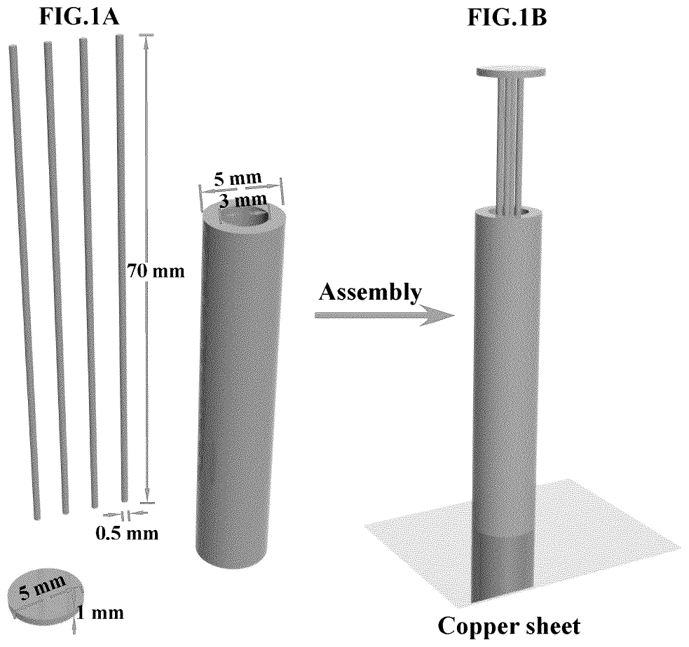

To get the multichannel design, a specific mold was fabricated by 3D printing technology. In addition to the traditional tube shell, 4 same pillars were set in the center. As shown in FIG. 1A, a cylinder with four sticks and a circular disc was printed. And according to FIG. 1B, the printed parts were assembled together. Copper sheets covered the bottom of the tube to achieve directional freeze.

### Embodiment 2. Fabrication of Aligned and Multichannel Conduit

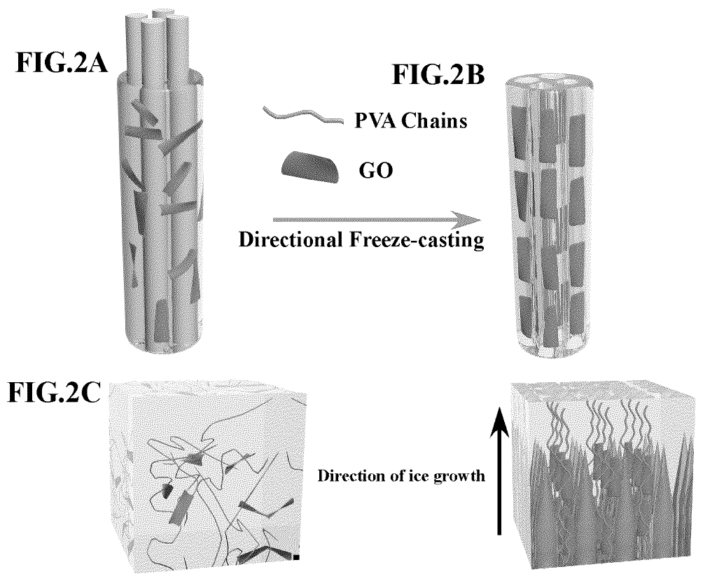

The preparation process of GO/PVA hydrogel conduit was illustrated in FIGS. 2A-2C. The 50 mg graphene oxide (GO) was prepared by a modified Hummers method and dispersed into 100 ml of deionized (DI) water. Then, 10 g of Polyvinyl alcohol (PVA) powder was added to the hot GO solution and stirred for 2 hours to ensure a homogenous solution. The prepared solution was injected into the designed model, followed by a directional freeze in that liquid nitrogen only made contact with copper sheets. After the frozen tube was freeze-dried, conduits were annealed at 100° C. for 30 minutes to fix the shape. All chemicals with analytical grade were purchased from Sigma-Aldrich without further treatment, and the DI water was used throughout the whole experiment.

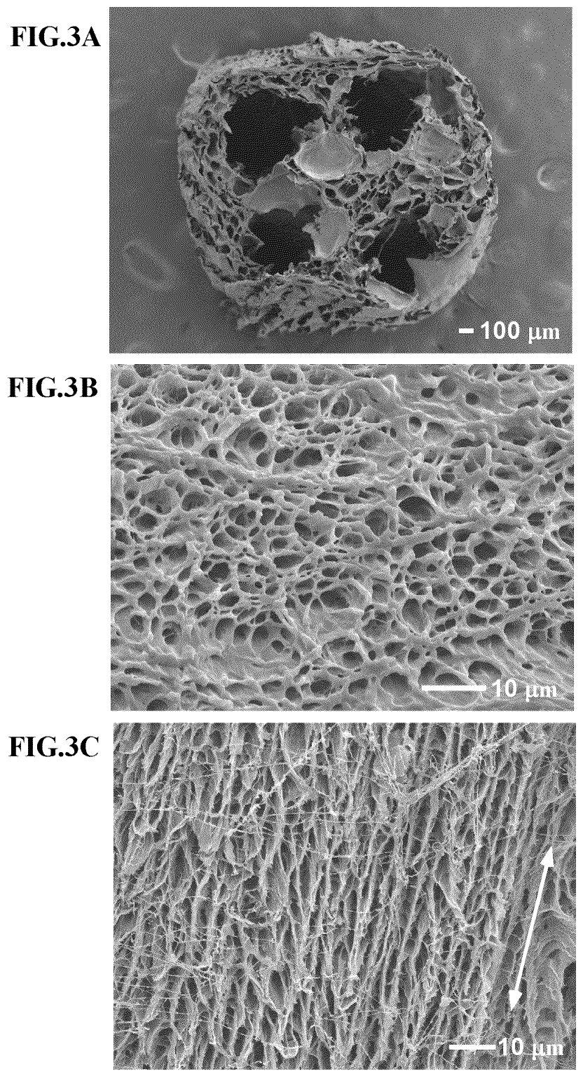

The morphology and structure of conduits were characterized using SEM. FIG. 3A illustrated the multichannel structure of the conduit, the four big pores are formed by the model. FIGS. 3B-3C proved the aligned structure of PVA fibers and GO sheets via directional freeze. All fibers were aligned in one direction in the longitudinal section of the conduit, and the cross-section of the conduit showed the existence of ice pillars.

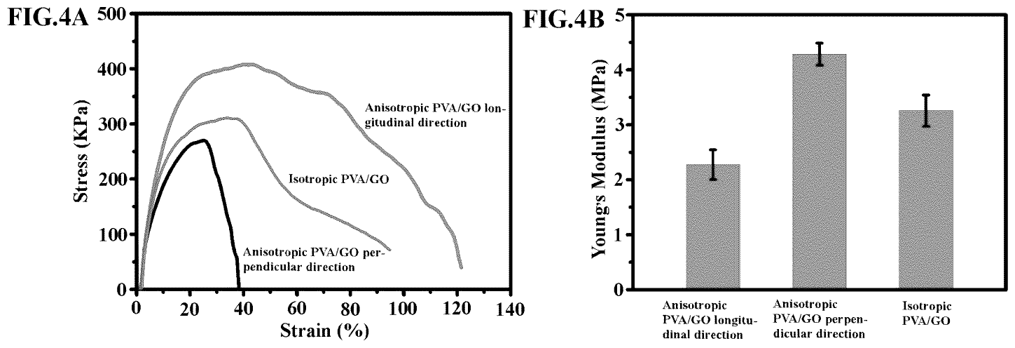

FIGS. 4A-4B illustrated the mechanical property of the conduit. Owing to the aligned arrangement of polymer chains, hydrogel showed anisotropic property, which was directly reflected by mechanical property. The Young's modulus, ultimate tensile strength, and elongation of conduit reached to maximum when the stretch direction was parallel to the freezing direction. However, when the stretch direction was perpendicular to the freezing direction, mechanical property of the conduit was weak. For isotropic PVA/GO hydrogel, the medium performance was revealed.

While certain embodiments have been illustrated and described, it should be understood that changes and modifications can be made therein in accordance with ordinary skill in the art without departing from the technology in its broader aspects as defined in the following claims.

The embodiments, illustratively described herein, may suitably be practiced in the absence of any element or elements, limitation, or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can of course vary. It is also to be understood that the terminology used herein is for the purpose of describing particular embodiments only, and is not intended to be limiting.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

What are claimed are:

1. A method of preparing an aligned and multichannel peripheral nerve guidance conduit, comprising:
preparing graphene oxide (GO) by a modified Hummers method and dispersing the GO into 100 ml of deionized (DI) water to obtain GO solution;
adding 10 g of Polyvinyl alcohol (PVA) powder into the GO solution and stirring for 2 hours to ensure a good mix;
injecting the prepared solution into the designed mold;
directional freezing that liquid nitrogen only contact with copper sheets, followed by freeze drying to obtain a dried sample; and
annealing the dried sample in a furnace at 100° C. for 30 minutes to fix the shape.

2. A designed mold by 3D printing and assembly, comprising:
one pipe (inner diameter: 3 mm, outer diameter: 5 mm, height: 50 mm);
four pillars (diameter: 0.5 mm, height: 70 mm); and
one disc (diameter: 5 mm, thickness: 1 mm);
wherein assembly steps thereof comprise:
gluing the pipe vertically to the copper sheet;
penetrating four cylinders vertically into a disc that the penetration points form a square (Side length: 2 mm) and the square and the disc's center coincide; and
putting four cylinders with a disc into the prepared pipe, and the disc can cover the whole pipe.
